(19) United States
(12) Patent Application Publication
(10) Pub. No.: US 2023/0372113 A1
(43) Pub. Date: Nov. 23, 2023

(54) DISC PROSTHESIS DEVICE FOR THE CERVICAL SPINE

(71) Applicant: COMPANION SPINE, LLC, NEW YORK, NY (US)

(72) Inventors: Vincent POINTILLART, BORDEAUX (FR); Jacques SENEGAS, MERIGNAC (FR); Richard ASSAKER, KIAN (BE); Richard MINFELDE, PARIS (FR)

(21) Appl. No.: 18/248,249

(22) PCT Filed: Sep. 21, 2021
(86) PCT No.: PCT/FR2021/051607

§ 371 Date: Apr. 7, 2023

(30) Foreign Application Priority Data
Oct. 9, 2020 (FR) FR2010361

(51) Int. Cl. A61F 2/44; A61F 2/30

(52) U.S. Cl. CPC A61F 2/441; A61F 2/442; A61F 2/30771; A61F 2002/30242; A61F 2002/3092; A61F 2002/3093

(57) ABSTRACT

Disc prosthesis comprising an insert in the form of a flexible cushion, a first half-shell and a second half shell, the insert in the form of a flexible cushion being positioned between the half-shells, characterized in that a first of the half-shells includes a first base provided with a first surface for hooking on to a first vertebra and a recessed annulus for forming a first cup and in that a second of the half-shells is provided with a first surface for hooking on to a second vertebra and a second surface bearing a rim surrounding a second cup, the cups facing each other each receiving an upper crown and a lower crown of the insert, the annulus and the rim forming walls that slot together with play and making stops for angular and shearing movements between the half-shells.

### CROSS REFERENCE TO RELATED APPLICATIONS

This application is a National Stage of International Application No. PCT/FR2021/051607 having an International Filing Date of 21 Sep. 2021, which designated the United States of America, and which International Application was published under PCT Article 21(2) as WO Publication No. 2022/074309 A1, which claims priority from and the benefit of French Patent Application No. 2010361, filed on 9 Oct. 2020, the disclosures of which are incorporated herein by reference in their entireties.

### BACKGROUND

### Field

The disclosure relates to the field of implantable vertebral disc prostheses and in particular to vertebral disc prostheses which are in particular adapted to the cervical spine.

### Brief Description of Related Developments

The wear suffered by the vertebral discs leads to the need for disc prostheses.

Disc prosthesis devices are known, such as an intervertebral disc prosthesis in the form of an inseparable structure which comprises a rigid upper plate, a rigid lower plate and an intermediate cushion, which is elastically compressible and housed between the internal surfaces of the two plates, the cushion having in the free state, that is to say without compressive stress, the shape of a wedge between the two plates. A disc prosthesis device is also known which comprises a flexible ring between two flat plates, the space at the center of the ring being filled with a gel or a liquid, or an intervertebral prosthesis comprising a disc-shaped bladder filled with a gel and surrounded by a flexible ring.

These devices offer few limits on the movements of inclination of the vertebrae with respect to an axis, connecting the vertebrae, perpendicular to the cushion, on the rotational movements about this axis, or on the shear movements according to the plane of the cushion. It is necessary to improve the disc prostheses by limiting the freedom of movement of the vertebrae to which the prosthesis is attached. Such a prosthesis device must also be easy to manufacture, even in small dimensions that make it usable as a cervical disc prosthesis, and it must be easy to implant. It is also desirable to be able to control the height of the prosthesis for its preparation and its implantation.

### SUMMARY

To do this, the present application proposes a disc prosthesis comprising an insert in the form of a flexible cushion, a first half-shell, a second half-shell, the insert in the form of a flexible cushion being positioned between said half-shells, wherein a first of the half-shells comprises a first base provided with a first face for attachment to a first vertebra and with a second face carrying a crown surrounding a first cup, and wherein a second of the half-shells is provided with a first face for attachment to a second vertebra and with a second face carrying a border surrounding a second cup, said cups facing each other and respectively receiving an upper cap and a lower cap of said insert, said crown and said border forming mutually facing inclined walls that fit together with a clearance (g) and produce stops on angular movements (a) and on shear movement (C1, C2) between the half-shells, said mutually facing inclined walls forming stops both in terms of proximity and inclination of one half-shell with respect to the other half-shell, said half-shells being held spaced apart by the insert during normal physiological stresses on the prosthesis.

The prosthesis of the disclosure, particularly well suited to forming a cervical disc prosthesis, is easily implantable, can be made in small sizes compatible with implantation at the level of the cervical vertebrae, and allows limited movements between these vertebrae.

The features set forth in the following paragraphs can optionally be implemented. They can be implemented independently of one another or in combination with one another.

Advantageously, said crown and said border comprise mutually facing walls that are inclined at complementary inclinations.

A foot of said border can be surrounded by a flange as a continuation of the first face of the second half-shell.

Said crown and said border can have a circular base, an oval base or a rectangular base with rounded corners.

With said crown and said border having a rectangular base with rounded corners, said corners advantageously form stops on rotation of one of the half-shells with respect to the other about an axis substantially perpendicular to the planes of the faces of attachment of the half-shells.

With the insert in position between the half-shells, the latter can be movable relative to each other in inclination with respect to an axis that is initially perpendicular to the first base and to the second base, by compression or deformation of at least part of an edge of the insert.

The insert in the form of a flexible cushion can be an insert of generally spheroidal shape with flattened or non-flattened polar caps.

The insert in the form of a flexible cushion can be hollow.

The hollow insert is advantageously filled with a gas, a liquid or a gel.

Preferably, the hollow insert is filled with an incompressible deformable material. The insert can also be solid and itself deformable and incompressible.

At least one of the bases can comprise a mesh structure and/or indentations, grooves or ridges, or it comprises a porous texture for attachment to the bone of the vertebra against which it is applied.

Said insert and said half-shells can be glued together, or they can be interlocked by having forms of complementary geometries.

### BRIEF DESCRIPTION OF THE DRAWINGS

Other features, details and advantages of the disclosure will become apparent on reading the detailed description below, and on analyzing the appended drawings, in which:

FIG. 1 shows an exploded view of a prosthesis according to a first embodiment;

FIG. 2 shows a cutaway perspective view of an example of an insert;

FIG. 3 shows a longitudinal sectional view of an embodiment of half-shells;

FIG. 4 shows a cutaway perspective view of a second embodiment of a prosthesis;

FIG. 5 shows a schematic view of the degrees of freedom of a prosthesis of the disclosure;

FIG. 6A shows a schematic sectional side view of an undeformed prosthesis according to the application;

FIG. 6B shows a schematic sectional side view of the prosthesis from FIG. 6A when angularly deformed.

### DETAILED DESCRIPTION

The drawings and the description of non-limiting examples of the disclosure may serve not only to permit a better understanding of the present disclosure but also to contribute to the definition thereof, where necessary.

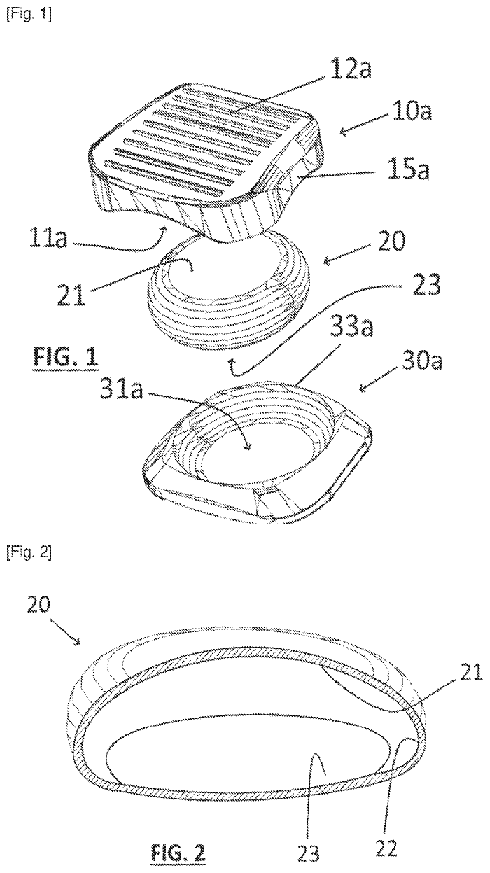

Reference is now made to FIG. 1 which shows an implant or disc prosthesis of the application in an exploded view according to a first embodiment. Said implant is a system which comprises a first half-shell 10a called the upper half-shell, an intersomatic element in the form of a flexible insert 20, and a second half-shell 30a called the lower half-shell.

The external faces of the half-shells constitute plates in contact with the vertebrae between which the implant is placed.

The flexible insert is received in mutually facing cups 11a, 31a formed in the half-shells.

The implant is more particularly suitable for producing a cervical disc prosthesis.

The flexible insert is advantageously a cushion as shown in FIG. 2. This insert can be spheroidal in shape, and its polar caps 21, 32, in contact with the inside of the half-shells, can be flattened or not flattened. The flexible insert can advantageously be hollow and comprise a flexible equatorial hoop.

The hollow insert is sealed and contains a gas, a liquid or a gel, depending on the flexibility that is to be given to it.

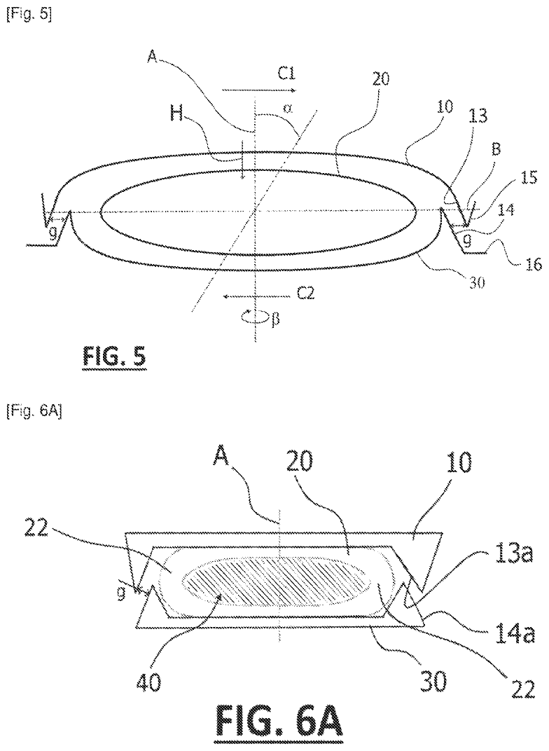

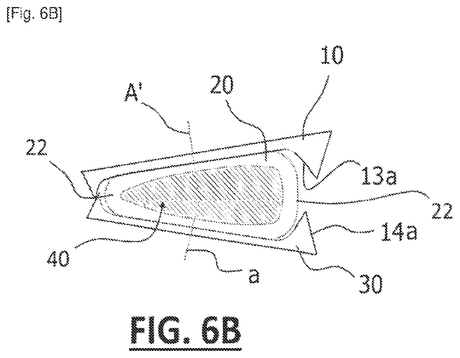

In a particular embodiment, the hollow flexible insert is filled with a liquid or an incompressible gel 40, and therefore its deformations will be effected by deformation of the hoop 22, as is shown in FIGS. 6A, 6B for example.

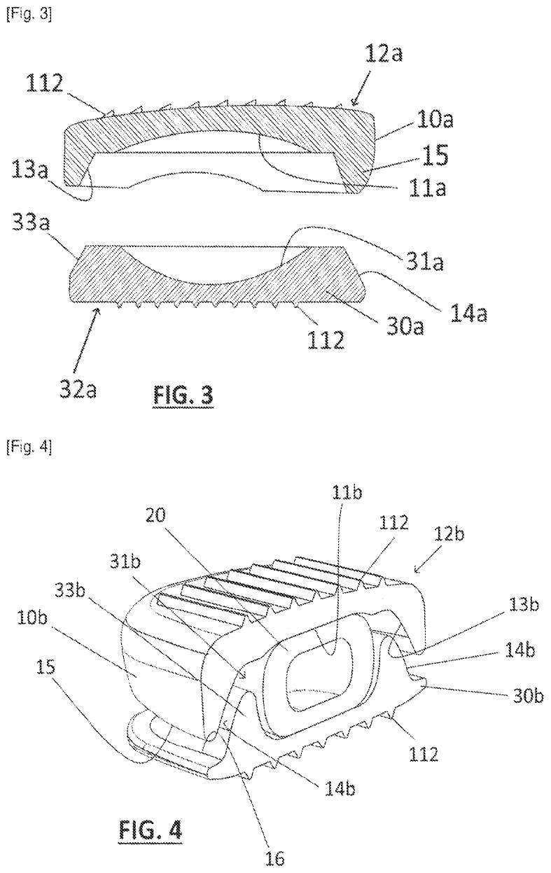

According to FIG. 3, for the embodiment of FIG. 1, the upper and lower half-shells comprise inclined walls 13, 14 forming stops with respect to both proximity and inclination of one half-shell relative to the other half-shell.

According to FIG. 4, which shows a second embodiment of half-shells of the disclosure, the upper half-shell 11b and the lower half-shell comprise abutment means formed by a lower edge 15 of the upper half-shell and by a flange 16 of the lower half-shell.

The benefit of a flexible insert arranged between two half-shells is that it approximates to the anatomy of a vertebral disc:
No fixed center of mobility in the form of a mechanical connection of the ball joint type or others;
The kinematics of the implant are essentially ensured by the flexible insert 20, called intersomatic insert.
The half-shells, once positioned between vertebrae, enclose the flexible insert and are movable in rotation about the flexible insert while forming stops.

The implant has damping properties by virtue of the flexible insert 20 which, arranged between the half-shells, permits non-rigid dynamic stabilization. By producing this hollow flexible insert with walls of variable thickness, the implant can be provided with variable rigidity, making it possible to adapt the stiffness of the insert according to different axes of rotation.

In this case, the flexibility of the flexible insert must be able to allow the natural movements of the head with respect to the chest and thus generate inclinations of an amplitude greater than 14° in flexion-extension and anteroposterior translation (shear) of at least 0.6 mm to 2 mm, depending on the size of the implant for cervical application. These are therefore significant deformation amplitudes of approximately 10 to 15%.

For this reason, the concept of play between the half-shells is essential in the disclosure, the play being such that in normal operation, that is to say within the ranges of physiological angulations and translations, the upper half-shell does not come into contact with the lower half-shell, the cushion ensuring balance thereof.

Furthermore, the shape of the half-shells is defined so as to allow easy and intuitive centering of the implant with respect to the plates at the treated level.

The crown 13a, 13b of the first half-shell and the border 33a, 33b of the second half-shell, which fit into each other with play, form a container for the flexible insert. The inner wall of the border and also the cup 11a form retaining walls for this implant.

The anchoring of the implant at the level of the vertebral plates is ensured by a structure promoting attachment to the vertebrae, for example a mesh structure which may or may not be supplemented with ribs 12 and/or studs in order to present good characteristics of osseointegration. It is also possible for the surface in contact with the bones to be given a porous texture, for example by chemical treatment or the addition of a layer of porous material. A hydroxyapatite coating can also be formed on the faces of the half-shells in contact with the vertebrae.

The half-shells can be produced from a 3D print modeled on the patient's anatomy. The material from which they are made can be based on titanium alloy, a material of the urethane type, PEEK (polyether ether ketone), a ceramic or any other biocompatible material that is sufficiently strong and adapted not to release particles during friction between the half-shells. The half-shells can also be produced using combinations of said materials, for example in order to produce an outer layer for attachment and an inner layer that is not especially sensitive to friction.

The range of implants is available in three or four sizes that are adapted to the morphologies of the vertebrae of the patients.

The materials of the implant are chosen so as to offer substantial radioparency and so as to offer very good compatibility with MRI imaging.

The shape and the dimensions of the implant make it possible to use an ancillary of small dimensions.

Both in the embodiment of FIG. 1 and in that of FIG. 3, one of the half-shells 10a, 10b is partially positioned in the other half-shell 30a, 30b with longitudinal play, lateral play and rotational play about the flexible insert.

In a direction of compression of the flexible insert, according to FIG. 3, the cup of the lower half-shell is surrounded by a border in the form of an inclined outer wall 33a on which there can bear an inclined inner wall 13a of a border of the upper cup. This constitutes a compression stop, which makes it possible to control the height of the prosthesis for its preparation and its implantation. Similarly, once implanted, an inclination of one of the half-shells with respect to the axis will be limited by the contact between the inclined walls.

In the case of FIG. 4, the stop effected during the compression of the insert takes place between the top 15 of the wall 10b and the flange 16, while the stop on inclination of the half-shells with respect to each other will take place on the inclined walls 13b, 33b.

FIG. 5 shows the various possible movements of the half-shells 10, 30 with respect to each other in the embodiment of FIG. 1. According to this figure, the walls 13, 14 are originally separated by a clearance g obtained by the fact that, with the upper half-shell resting on the insert, the sloping walls 13, 14 of the crown and of the border are spaced from each other by the insert. Firstly, an inclination of one of the half-shells at an angle α with respect to the vertebral axis A is possible, but it is limited by the abutment on one side or the other of the walls 13, 14.

The shear movement C1, C2 is for its part also limited by the clearance g between the walls 13, 14. The rotational movement β of one of the shells with respect to the other about the axis A is itself limited by the general rectangular shape of the half-shells, as seen in FIG. 1. In the case of the embodiment of FIG. 4 and of a compression movement in the direction H, the free top of the crown 15 comes into abutment on the flange 16. Similarly, in the case of the embodiment of FIG. 3, the wall 33b or its top can come into abutment with the internal wall 13b of the cup of the lower half-shell 10. However, these contacts only occur in the event of abnormal compression or failure of the insert as a safety means.

FIGS. 6A and 6B show the deformation of the cushion 20 between the half-shells during an inclination of one of the half-shells with respect to the other, still in the case of the example of FIG. 3.

In the case of the embodiments of FIGS. 3 and 4, a shear movement of one of the half-shells with respect to the other will be limited by the contact of the walls 13a, 13b, 33a, 33b.

The half-shells and the tops of the flexible insert can be glued together to form a one-piece disc, which is easier to implant.

For a vertebral implant in the form of the cervical disc prosthesis, three to four implant sizes can be provided, and the dimensions of this implant can be of the order of:
Small size: width of the order of 12 to 14 mm, length of the order of 14 to 16 mm;
Medium size: width of the order of 14 to 16 mm, length of the order of 16 to 18 mm;
Large size: width of the order of 16 to 18 mm, length of the order of 18 to 20 mm.
   The heights of the implants can be in four sizes of the order of 4 to 5 mm, 5 to 6 mm, 6 to 7 mm or 7 to 8 mm depending on the morphology of the patients who are going to receive them.

The half-shells can be of a two-component type, with:
a part provided for contact with the vertebrae and made of titanium alloy for good integration in the vertebral plates. A certain porosity (not shown) can also be used for recolonization, produced by additive manufacturing;
parts in contact with the cushion and in contact with each other during movement, made of polymer such as PEEK, PEKK, polyethylene in order to offer low abrasion.

It is also possible to envision single-component half-shells, in which case one would opt for a ceramic or even titanium alloy, but with a coating making it possible to strengthen and improve the wear properties of the central parts, or finally any polymer, with the surfaces of the plates which are in contact with the bone being coated with materials conducive to osteogenesis.

The cushion can be made of silicone, polyurethane, or deformable polymer and, once positioned in the half-shells, must permit in combination with the latter a flexion-extension mobility of the order of 18° combined with antero-posterior translation variable according to the sizes, in particular an amplitude of approximately 1 mm to 2 mm, a lateral inclination of the order of 15° and a rotation of at least 10° according to the clearance g obtained when the half-shells are positioned on the cushion and are thus spaced apart from each other during normal physiological stresses.

The disclosure is not limited to the examples which are described above only by way of example, and instead it encompasses all the variants that a person skilled in the art may consider within the scope of the protection sought. In particular, the terms upper half-shell and lower half-shell are used only for convenience in order to make the text easier to read, it being understood that the insert can also be positioned upside down.

What is claimed is:

1. A disc prosthesis comprising an insert in the form of a flexible cushion, a first half-shell, a second half-shell, the insert in the form of a flexible cushion being positioned between the half-shells, characterized in that a first of the half-shells comprises a first base provided with a first face for attachment to a first vertebra and with a second face carrying a crown surrounding a first cup, and wherein a second of the half-shells is provided with a first face for attachment to a second vertebra and with a second face carrying a border surrounding a second cup, the cups facing each other and respectively receiving an upper cap and a lower cap of the insert, the crown and the border forming mutually facing inclined walls that fit together with a clearance and producing stops on angular movements and shear movement between the half-shells, the mutually facing inclined walls forming stops both on proximity and inclination of one half-shell with respect to the other half-shell, the half-shells being held spaced apart by the insert during normal physiological stresses on the prosthesis.

2. The disc prosthesis as claimed in claim 1, wherein the crown and the border comprise mutually facing walls inclined at complementary inclinations.

3. The disc prosthesis as claimed in claim 1, wherein a foot of the border is surrounded by a flange as a continuation of the first face of the second half-shell.

4. The disc prosthesis as claimed in claim 1, wherein the crown and the border have a circular base, an oval base or a rectangular base with rounded corners.

5. The disc prosthesis as claimed in claim 4, wherein the crown and the border have a rectangular base with rounded corners, the corners forming stops on rotation of one of the half-shells with respect to the other about an axis substantially perpendicular to the planes of the attachment faces of the half-shells.

6. The disc prosthesis as claimed in claim 1, wherein, with the insert in position between the half-shells, the latter are movable relative to one another in inclination with respect to an axis initially perpendicular to the first base and to the second base by compression or deformation of at least part of an edge of the insert.

7. The disc prosthesis as claimed in claim 1, wherein the insert in the form of a flexible cushion is an insert of generally spheroidal shape with polar caps, which are flattened or not flattened.

8. The disc prosthesis as claimed in claim 1, wherein the insert in the form of a flexible cushion is hollow.

9. The disc prosthesis as claimed in claim 8, wherein the hollow insert is filled with a gas, a liquid or a gel.

10. The disc prosthesis as claimed in claim 8, wherein the hollow insert is filled with an incompressible deformable material.

11. The disc prosthesis as claimed in claim 1, wherein at least one of the bases comprises a mesh structure and/or indentations, grooves, ridges, or comprises a porous texture for attachment to the bone of the vertebra against which it is applied.

12. The disc prosthesis as claimed in claim 1, wherein the insert and the half-shells are glued together, or they are interlocked by having shapes of complementary geometries.
